(19) United States
(12) Patent Application Publication
(10) Pub. No.: US 2013/0137922 A1
(43) Pub. Date: May 30, 2013

(54) Endoscope

(71) Applicants: Yen-Nien CHEN, Kaohsiung City (TW); Wei-Jen SHIH, Kaohsiung City (TW); Wei-The CHEN, Kaohsiung (TW); Cheng-Wen LIN, Kaohsiung (TW); Pei-Yuan LEE, Kaohsiung (TW)

(72) Inventors: Yen-Nien CHEN, Kaohsiung City (TW); Wei-Jen SHIH, Kaohsiung City (TW); Wei-The CHEN, Kaohsiung (TW); Cheng-Wen LIN, Kaohsiung (TW); Pei-Yuan LEE, Kaohsiung (TW)

(21) Appl. No.: 13/592,405

(22) Filed: Aug. 23, 2012

(30) Foreign Application Priority Data
Nov. 24, 2011 (TW) 100143086

(51) Int. Cl. A61B 1/005; A61B 1/06; A61B 1/01; A61B 1/04

(52) U.S. Cl. 600/109

(57) ABSTRACT

An endoscope includes a tube having including a compartment extending in a longitudinal direction. A flexible strip has a diameter smaller than a diameter of the compartment. The flexible strip is received in the compartment and slideable relative to the tube in the longitudinal direction. The flexible strip is made of a super elastomer having an original shape before deformation. The flexible strip is capable of restoring the original shape after larger deformation. An image capturing module is mounted to the flexible strip. The image capturing module can transmit light beam and capture images.

### BACKGROUND OF THE INVENTION

1. Field of the Invention

The present invention relates to an endoscope and, more particularly, to an endoscope that can be inserted into a human body without using natural orifices of the human body, with the endoscope suitable for joints or the celia of the human body.

2. Description of the Related Art

FIG. 1 shows a conventional endoscope 9 allowing rotation in two or four directions. The endoscope 9 includes a plurality of spaced hollow rotary members 91 each having a plurality of through-holes 911 (FIG. 1a) and two connecting faces 912. The endoscope 9 further includes a plurality of connecting members 92 (FIG. 1b) each having two ends respectively connected to two mutually facing connecting faces 912 respectively of two adjacent rotary members 91. The endoscope 9 further includes a plurality of guiding lines 93 extending through the through-holes 911 of the hollow rotary members 91. By pulling one of the guiding lines 93, the plurality of rotary members 91 can be bent in either of two directions. An example of such an endoscope 9 is disclosed in Taiwan Utility Model No. M400299. To obtain a certain bending effect of the endoscope 9, each rotary member 91 must be small for the purposes of increasing the number of the serially connected rotary members 91. However, the difficulties in formation of the through-holes 911 in each rotary member 91 and connection of each connecting member 92 between two adjacent rotary members 91 will be aggravated by the decrease in the size of the rotary members 91 and lead to an increase in the manufacturing costs.

Furthermore, operation for bending the rotary members 91 in the desired direction requires pulling of the corresponding guiding line 93 and precise control of the magnitude of force pulling the guiding line 93, which is not easy. Further, the number of the guiding lines 93 has to be increased if the number of the directions to be controlled is increased, complicating the operation. Namely, an operator has to spend more time practicing pulling of the correct guiding line 93 and controlling the force for pulling the guiding line 93 so as to move the endoscope 9 to an ideal position in the cavity of the human body for capturing images.

### SUMMARY OF THE INVENTION

The term “super elastomer” used herein relates to a material including a Young's modulus having a non-linear stress-strain curve.

An objective of the present invention is to provide an endoscope to solve the disadvantages in the prior art, with the endoscope including a flexible strip made of a material having super elastomericity to provide simplify the structure and to reduce the manufacturing costs.

Another objective of the present invention is to provide an endoscope that is simple in structure and easy to operate.

The present invention fulfills the above objectives by providing an endoscope including a tube having including a compartment extending in a longitudinal direction. A flexible strip has a diameter smaller than a diameter of the compartment. The flexible strip is received in the compartment and slideable relative to the tube in the longitudinal direction. The flexible strip is made of a super elastomer having an original shape before deformation. The flexible strip is capable of restoring the original shape after larger deformation. An image capturing module is mounted to the flexible strip. The image capturing module is adapted to transmit light beams and to capture images.

Preferably, the tube is made of a rigid material that is difficult to deform.

Preferably, the flexible tube includes a front end and a rear end. The image capturing module includes a photosensitive chip and at least one signal line. The photosensitive chip is mounted to the front end of the flexible tube. The at least one signal line is electrically connected to the photosensitive chip.

In an example, the flexible strip includes an outer periphery having at least one groove. The at least one signal line is received in the at least one groove. A sheath is mounted around the outer periphery of the flexible strip.

In another example, the flexible tube is a hollow tube defining a receiving space, and the at least one signal line is received in the receiving space.

Preferably, a controller is mounted to the endoscope and connected to the flexible strip. The controller is operable to move the flexible strip relative to the tube.

Preferably, the controller includes a sliding groove and a control member slideably received in the sliding groove. The body is fixed to the tube, with the sliding groove in communication with the compartment, and with the control member connected to the flexible strip.

Preferably, the flexible strip is made of a shape memory alloy or polymer.

Preferably, the flexible strip is made of a nickel-titanium alloy.

The present invention will become clearer in light of the following detailed description of illustrative embodiments of this invention described in connection with the drawings.

### BRIEF DESCRIPTION OF THE DRAWINGS

The illustrative embodiments may best be described by reference to the accompanying drawings where:

FIG. 1 shows a perspective view of a conventional endoscope.

FIG. 1a shows an enlarged view of a circled portion of FIG. 1.

FIG. 1b shows an enlarged view of another circled portion of FIG. 1.

FIG. 2 shows an exploded, perspective view of an endoscope according to the present invention.

FIG. 3a shows a cross sectional view of an example of a flexible strip of the endoscope according to the present invention.

FIG. 3b shows a cross sectional view of another example of the flexible strip of the endoscope according to the present invention.

FIG. 4 shows a partial, perspective view of an endoscope of another embodiment according to the present invention.

FIG. 5a shows a partial, cross sectional view of the endoscope of FIG. 4, with a control member located in an end of a sliding groove.

FIG. 5b shows a view similar to FIG. 5a, with the control member located in an intermediate portion in the sliding groove.

FIG. 5c shows a view similar to FIG. 5a, with the control member located in the other end of the sliding groove.

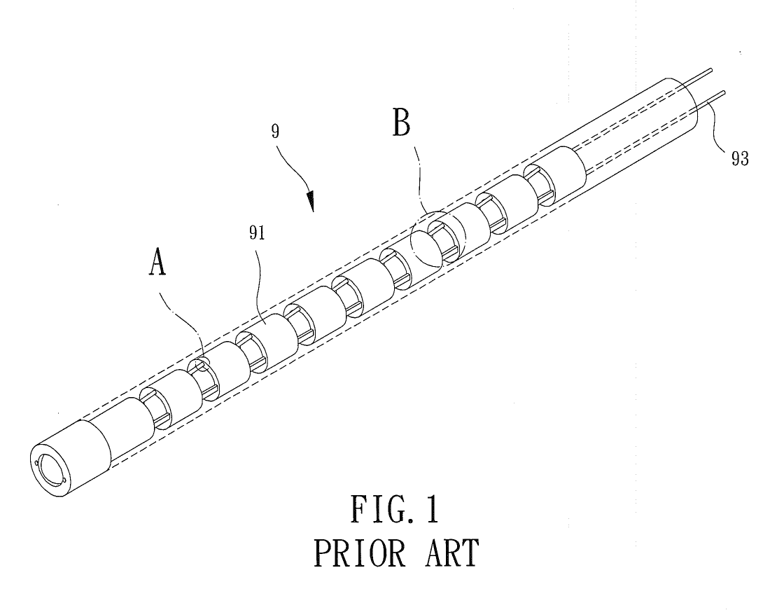

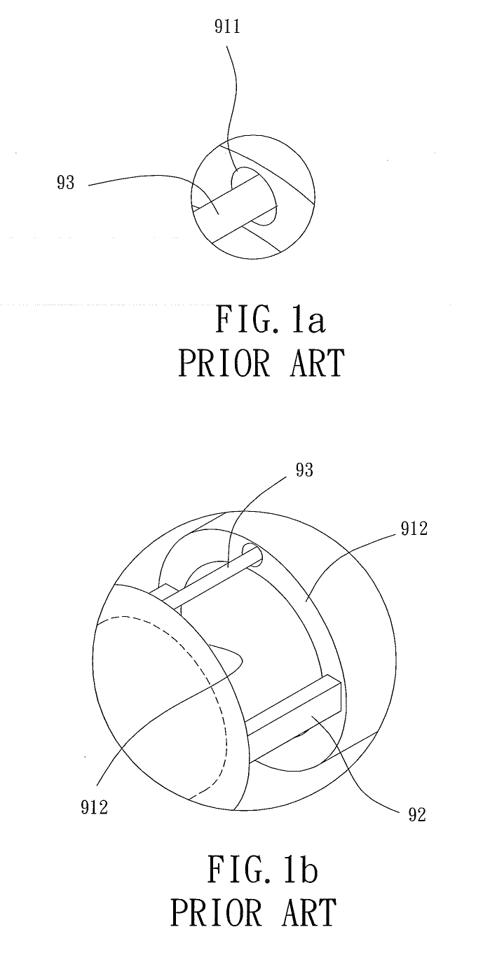

All figures are drawn for ease of explanation of the basic teachings of the present invention only; the extensions of the figures with respect to number, position, relationship, and dimensions of the parts to form the preferred embodiments will be explained or will be within the skill of the art after the following teachings of the present invention have been read and understood. Further, the exact dimensions and dimensional proportions to conform to specific force, weight, strength, and similar requirements will likewise be within the skill of the art after the following teachings of the present invention have been read and understood.

### DETAILED DESCRIPTION OF THE INVENTION

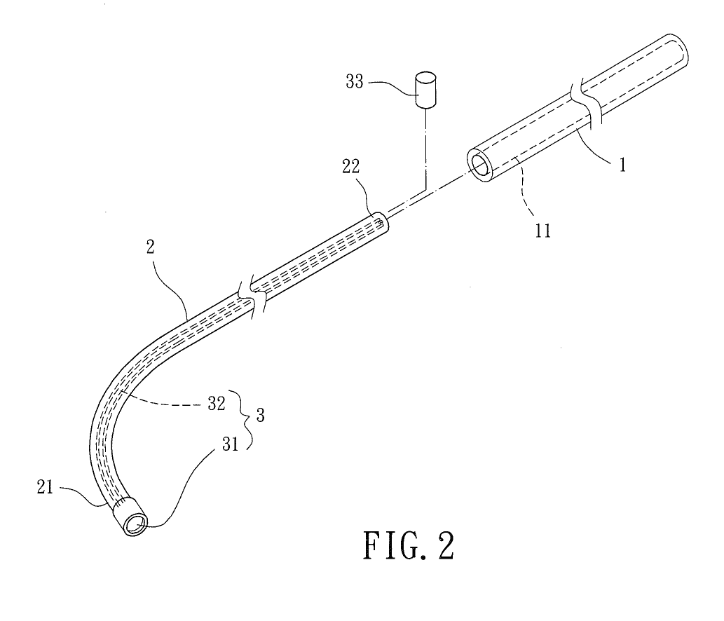

FIG. 2 shows an endoscope of an embodiment according to the present invention. The endoscope is suitable for use in joints or the celia of a human body. The endoscope includes a tube 1, a flexible strip 2 received in the tube 1, and an image capturing module 3 received in the flexible strip 2. Tube 1 restrains bending of the flexible strip 2.

The tube 1 includes a compartment 11 extending through the tube 1 in a longitudinal direction. In this embodiment, the tube 1 is preferably formed of rigid material (such as biomedical grade stainless steel or ceramic) that is difficult to deform, allowing the endoscope according to the present invention to be inserted into a joint or the celia through easy operation and allowing control of the depth and angle of insertion.

The flexible strip 2 includes a front end 21 and a rear end 22. The flexible strip 2 is made of a super elastomer that can withstand large deformation and that can restore its original shape before deformation after the load exerting on the flexible strip 2 is released. The flexible strip 2 can be made of a shape memory alloy (such as a nickel-titanium alloy) or a polymer (such as rubber). In this embodiment, the flexible strip 2 has a predetermined sinuosity. Furthermore, the flexible strip 2 has a diameter smaller than a diameter of the compartment 11. Thus, the flexible strip 2 deforms according to the shape of the tube 1 and is received in the tube 1. By pulling the rear end 22 of the flexible strip 2, the flexible strip 2 can slide in the tube 1 to control an extended length of the front end 21 of the flexible strip 2 outside out of the tube 1.

The image capturing module 3 includes a photosensitive chip 31 and at least one signal line 32. The photosensitive chip 31 is mounted in the front end 21 of the flexible strip 2 and electrically connected to the at least one signal line 32. In this embodiment, a plurality of signal lines 32 is used to transmit light beams to the front end 21 of the flexible strip 2 for illumination purposes. Thus, the photosensitive chip 31 can clearly send an image signal through the signal lines 32 to a processing unit which converts the image signal into a picture, and the picture is displayed on a display unit.

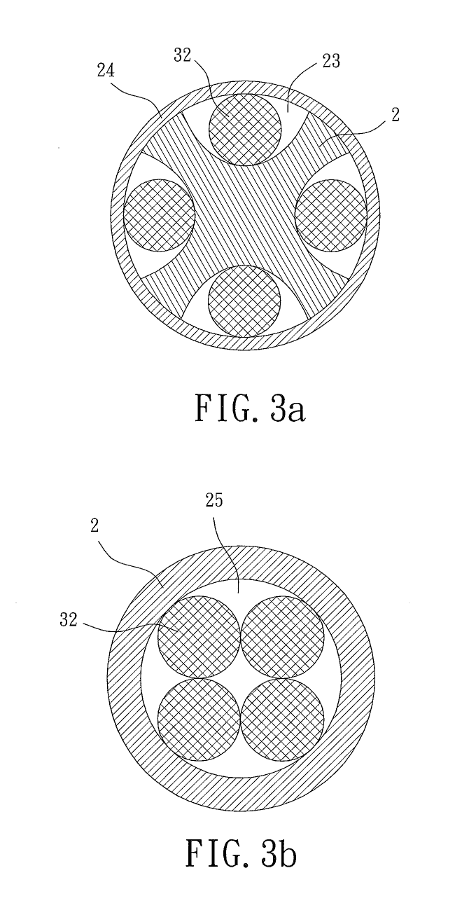

In an example shown in FIG. 3a, the flexible strip 2 includes an outer periphery having a plurality of grooves 23 each receiving a signal line 32. Preferably, a sheath 24 is mounted around the outer periphery of the flexible strip 2. After the signal lines 32 are inserted into the grooves 23, the sheath 24 is mounted around the flexible strip 2 to reliably position each signal line 32 in the corresponding groove 23 without the risk of falling.

In another example shown in FIG. 3b, the flexible strip 2 is in the form of a hollow tube defining a receiving space 25 in which the at least one signal line 32 is received.

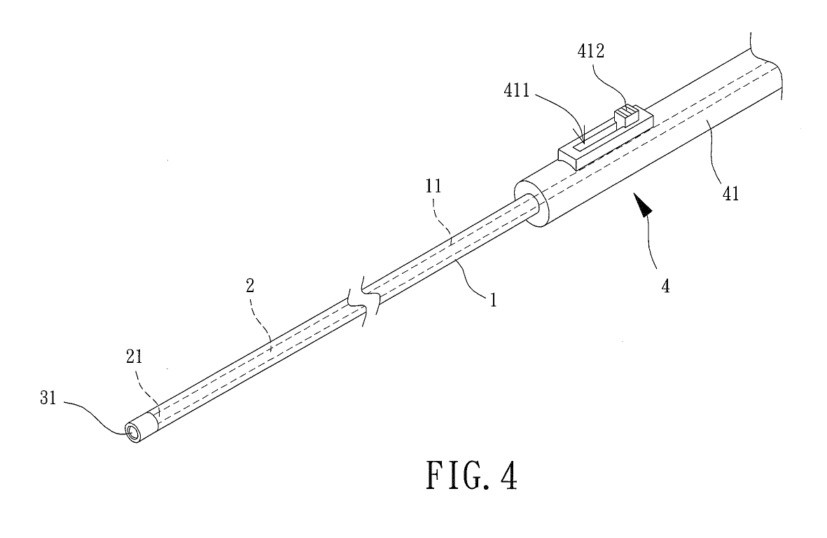

With reference to FIG. 4, to enhance the operational convenience, the endoscope can further include a controller 4 that is mounted to an end of the tube 1. The controller 4 can move the flexible strip 2 in the compartment 11 to control the extended length of the front end 21 of the flexible strip 2 outside of the tube 1. In this embodiment, the controller 4 includes a hollow body 41 through which the flexible strip 2 extends. The body 41 includes a sliding groove 411 and a control member 412 slideably received in the sliding groove 411. The body 41 is fixed to the tube 1, with the sliding groove 411 in communication with the compartment 11, and with the control member 412 connected to the flexible strip 2.

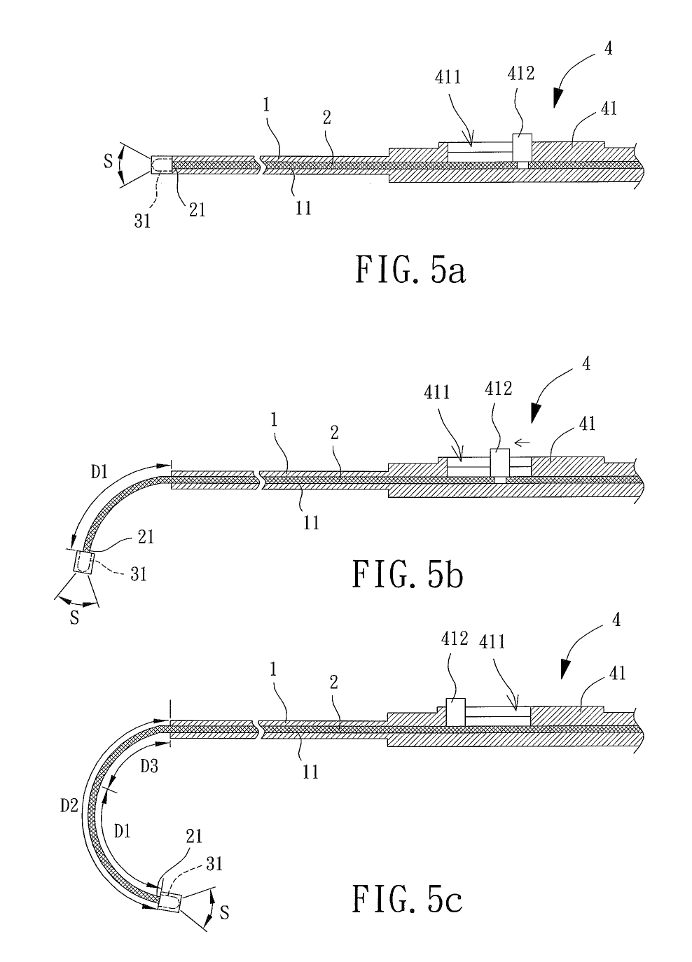

With reference to FIG. 5a, in use, the control member 412 is initially in a first position in the sliding groove 411 (an end of the sliding groove 411 having a spacing to the front end 21 of the flexible strip 2 larger than the other end of the sliding groove 411). The flexible strip 2 is completely received in the compartment 11 of the tube 1. However, the front end 21 of the flexible strip 2 can slightly extend out of the compartment 11. After an operator inserts the tube 1 of the endoscope into a cavity of a human body, the photosensitive chip 31 captures an image and sends an image signal indicative of the image through the signal lines 32 to the processing unit that converts the image signal into a picture and displays the picture on the display unit. Thus, the operator can see the interior image of the cavity in a visible range S.

With reference to FIG. 5b, if it is desired to change the visual angle of the endoscope, the control member 412 can be moved to a second position between two ends of the sliding groove 411. The flexible strip 2 is moved relative to the compartment 11 in the longitudinal direction, and the front end 21 of the flexible strip 2 extends out of the tube 1. The extended portion of the flexible strip 2 outside of the tube 1 can deform and bend, with the front end 21 of the flexible strip 2 having an arcuate spacing D1 to the front end of the tube 1. The image capturing direction of the front end 21 of the flexible strip 2 is, thus, changed.

With reference to FIG. 5c, when the control member 412 is moved to a third position in the other end of the sliding groove 411, the front end 21 of the flexible strip 2 is further extended out of the tube 1. The extended portion of the flexible strip 2 outside of the tube 1 restores its sinuosity, with the front end 21 of the flexible strip 2 having an arcuate spacing D2 to the front end of the tube 1. The arcuate spacing D2 is larger than the arcuate spacing D1 by an arcuate spacing D3. The image capturing direction of the front end 21 of the flexible strip 2 is changed again by restoration of the shape of the flexible strip 2. In the example shown, the image capturing direction of the front end 21 of the flexible strip 2 is at an angle of almost 180° to that of the front end 21 of the flexible strip 2 in FIG. 5a.

Thus, by changing the extended length of the front end 21 of the flexible strip 2 outside of the tube 1 and by rotating the tube 1, the visual angle of the endoscope can be changed arbitrarily. Thus, the endoscope has no dead angles in obtaining the images, enhancing the utility of the endoscope.

After capturing the images, the control member 412 is moved back to the first position in the sliding groove 411, and the flexible strip 2 deforms and is retrieved into the compartment 11 of the tube 1. Then, the operator can remove the tube 1 of the endoscope from the cavity of the human body.

According to the foregoing, the endoscope according to the present invention has a flexible strip 2 that is simple in structure by using the material characteristics of shape memory alloys or polymers having excellent elastomericity, significantly reducing the manufacturing costs.

In the endoscope according to the present invention, the extended length of the front end 21 of the flexible strip 2 can be controlled by simply moving the flexible strip 2 to arbitrarily change the visual angle of the endoscope. Thus, the endoscope has no dead angles in obtaining the images, enhancing the utility of the endoscope.

Thus since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

What is claimed is:

1. An endoscope comprising:
a tube including a compartment extending in a longitudinal direction of the tube;
a flexible strip having a diameter smaller than a diameter of the compartment, with the flexible strip received in the compartment and slideable relative to the tube in the longitudinal direction, with the flexible strip being made of a super elastomer having an original shape before deformation, with the flexible strip being made of the super elastomer capable of restoring the original shape after larger deformation; and
an image capturing module mounted to the flexible strip, with the image capturing module adapted to transmit light beams and to capture images.

2. The endoscope as claimed in claim 1, with the tube made of a rigid material that is difficult to deform.

3. The endoscope as claimed in claim 1, with the flexible tube including a front end and a rear end, with the image capturing module including a photosensitive chip and at least one signal line, with the photosensitive chip mounted to the front end of the flexible tube, with the at least one signal line electrically connected to the photosensitive chip.

4. The endoscope as claimed in claim 3, with the flexible strip including an outer periphery having at least one groove, with the at least one signal line received in the at least one groove.

5. The endoscope as claimed in claim 4, further comprising: a sheath mounted around the outer periphery of the flexible strip.

6. The endoscope as claimed in claim 3, with the flexible tube being a hollow tube defining a receiving space, with the at least one signal line received in the receiving space.

7. The endoscope as claimed in claim 1, further comprising: a controller mounted to the endoscope and connected to the flexible strip, with the controller operable to move the flexible strip relative to the tube.

8. The endoscope as claimed in claim 7, with the controller including a hollow body through which the flexible strip extends, with the body including a sliding groove and a control member slideably received in the sliding groove, with the body fixed to the tube, with the sliding groove in communication with the compartment, and with the control member connected to the flexible strip.

9. The endoscope as claimed in claim 1, with the flexible strip made of a shape memory alloy or polymer.

10. The endoscope as claimed in claim 1, with the flexible strip made of a nickel-titanium alloy.
